(19) United States
(12) Patent Application Publication
(10) Pub. No.: US 2026/0053984 A1
(43) Pub. Date: Feb. 26, 2026

(54) TISSUE ENGINEERING CARTILAGE CONSTRUCTED BASED ON DECALCIFIED BONE SCAFFOLD AND USE THEREOF

(71) Applicants: SHANGHAI BO ZHAO EN BIOTECHNOLOGY CO., LTD., Shanghai (CN); SHANGHAI NINTH PEOPLE'S HOSPITAL, SHANGHAI JIAOTONG UNIVERSITY SCHOOL OF MEDICINE, Shanghai (CN)

(72) Inventors: Guangdong ZHOU, Shanghai (CN); Yu LIU, Shanghai (CN); Zheng CI, Shanghai (CN)

(21) Appl. No.: 19/104,784

(22) PCT Filed: Aug. 21, 2023
(86) PCT No.: PCT/CN2023/114042

§ 371 Date: Feb. 19, 2025

(30) Foreign Application Priority Data
Aug. 19, 2022 (CN) 202210999824.9

(51) Int. Cl. A61L 27/36; A61L 27/38; C12N 5/077; C12N 5/0775

(52) U.S. Cl. CPC A61L 27/3608; A61L 27/365; A61L 27/3654; A61L 27/3834; A61L 27/3847; A61L 27/3852; C12N 5/0655; C12N 5/0663; A61L 2430/02; A61L 2430/06; A61L 2430/24; A61L 2430/40; C12N 2500/25; C12N 2500/32; C12N 2500/34; C12N 2500/38; C12N 2501/105; C12N 2501/15

(57) ABSTRACT

A tissue engineering cartilage constructed based on a decalcified bone scaffold, a preparation method therefor, and use thereof in the repairing of joint defects are provided. A marrow fluid of a patient is extracted, bone marrow mesenchymal stem cells of the patient are isolated and amplified in vitro, and the bone marrow mesenchymal stem cells isolated and cultured in vitro are inoculated into a decalcified bone scaffold, thus constructing a tissue engineering cartilage in vitro. The tissue engineering cartilage can be transplanted to a joint defect part, so that the effective repair of joint defects and function reconstruction are realized.

### TECHNICAL FIELD

The present invention relates to the field of biomedical tissue engineering, and in particular, to a tissue engineering cartilage constructed based on a decalcified bone scaffold, a preparation method therefor, and use thereof in the repairing of joint defects.

### BACKGROUND

Osteoarthritis (OA) is the most common degenerative joint disease, which has an extremely high incidence rate among the middle-aged and elderly people, and affects a large number of people. At the time of onset, the patients suffer from severe pain in the affected joints, limited mobility, and basic loss of labor ability, which seriously affects their daily life and work quality, causing a heavy burden on many families. It has become a major social problem affecting the quality of life of middle-aged and elderly people. Therefore, the research value of osteoarthritis prevention and treatment is enormous. Its effective prevention and treatment can significantly improve the life quality of patients, reduce social burden, and bring huge social and economic benefits. At present, the clinical treatment measures for osteoarthritis vary greatly depending on the degree of cartilage degeneration. For early-stage patients with mild degeneration, conservative treatments such as hormone anti-inflammatory therapy, hyaluronic acid lubrication, and platelet rich plasma (PRP) are commonly used. These methods can alleviate local symptoms, but cannot prevent the continuous degeneration of articular cartilage. For patients with moderate degeneration and localized cartilage defects, microfracture surgery and autologous bone cartilage transplantation are mainly used. The former mainly relies on bone marrow mesenchymal stem cells gushing out from subchondral bone to regenerate a small amount of fibrous cartilage, but it is only limited to a very small range of cartilage defects, and has unsatisfactory long-term effects; The latter may cause significant trauma to the donor site, and has limited donor sources, with poor integration between the transplanted cartilage and the surrounding normal cartilage (Mosaic phenomenon). For patients with severe degeneration and extensive destruction of articular cartilage, artificial joint replacement surgery is mainly used to replace joint structure and function. However, currently commonly used artificial joints based on metal, ceramics, etc., are expensive, lack biological function, and are prone to cause infection and foreign body rejection. They also have drawbacks such as long-term wear and prosthesis loosening, and often require secondary or even multiple revisions. Overall, the above treatment methods have their own limitations and cannot achieve physiological, permanent joint function reconstruction. How to achieve biological joint regeneration and permanent functional reconstruction that truly conform to physiological structure is still an insurmountable international challenge.

In recent years, with the advancement of tissue engineering, people have gradually begun to study the use of scaffolds or tissues constructed by tissue engineering for the attempt to repair joint defects. It has been confirmed by researches that for early-stage patients with mild degeneration, the use of autologous stem cells combined with anti-inflammatory, immune regulatory, and other measures can effectively prevent the progression of OA and even partially reverse cartilage degeneration. For moderate degeneration or cartilage injury with localized cartilage defects, autologous chondrocyte transplantation or combined transplantation with degradable scaffolds such as collagen and gelatin can also achieve a certain degree of cartilage regeneration and significantly improve clinical symptoms. However, this technology is limited to the repair of localized cartilage defects and has poor effects on the bone phase repair and cartilage-bone interface integration for combined bone defects. At the same time, this technology requires open surgical transplantation, which results in longer surgery time and slower postoperative recovery for patients. In addition, the trauma caused by obtaining chondrocytes on damaged joints, the success rate of cartilage regeneration after cell-material composite transplantation, and the limited mechanical properties of chondrocytes also greatly limit its clinical application. Therefore, at present, there is still a lack of an effective treatment method for joint defects.

### SUMMARY OF THE INVENTION

The objective of the present invention is to provide a tissue engineering cartilage constructed based on a decalcified bone scaffold, a preparation method therefor, and use thereof in the repairing of joint defects.

In the first aspect of the present invention, it provides a tissue engineering cartilage, which comprises:
(a) a carrier scaffold comprising a decalcified bone scaffold; and
(b) bone marrow mesenchymal stem cells inoculated into or loaded onto the carrier.

In another preferred embodiment, the tissue engineering cartilage comprises a complex formed by inoculating the bone marrow mesenchymal stem cells into the carrier and subjecting them to chondrogenic culture; in the complex, the bone marrow mesenchymal stem cells are loaded onto the carrier and form a tighter integrated structure with the carrier.

In another preferred embodiment, the bone marrow mesenchymal stem cells are derived from a human or a non-human mammal.

In another preferred embodiment, the bone marrow mesenchymal stem cells are isolated from the autologous bone marrow fluid of a subject.

In another preferred embodiment, the subject is a human or a non-human mammal.

In another preferred embodiment, the subject suffers from joint defects or other types of hard tissue defects or deformities.

In another preferred embodiment, the joint defects comprise cartilage defects, hard bone defects, or combinations thereof.

In another preferred embodiment, the joint defect is a knee joint defect, an elbow joint defect, a hip joint defect, an ankle joint defect, a wrist joint defect, a mandibular joint defect, or a combination thereof.

In another preferred embodiment, the other types of hard tissue defects or deformities include, but are not limited to, tibial defects, femoral defects, humeral defects, mandibular deformities, and zygomatic deformities.

In another preferred embodiment, the bone marrow mesenchymal stem cells are bone marrow mesenchymal stem cells cultured in vitro to P2 to P5 passages, preferably P3 passage bone marrow mesenchymal stem cells.

In another preferred embodiment, the inoculation density of the bone marrow mesenchymal stem cells into the carrier is 20-100×10⁶ cells/cm³, preferably 40-80×10⁶ cells/cm³.

In another preferred embodiment, the decalcified bone scaffold comprises an allogeneic decalcified bone scaffold, a heterologous decalcified bone scaffold, and a composite scaffold constructed with decalcified bone as the main structure.

In another preferred embodiment, the shape of the decalcified bone scaffold includes a cylinder, a cuboid, or other specific shapes.

In another preferred embodiment, the decalcified bone scaffold is a cylinder, with a diameter of 4-8 mm and a height of 6-10 mm.

In another preferred embodiment, the pore diameter of the decalcified bone scaffold is 200-400 μm, with a porosity of 80% to 90%.

In another preferred embodiment, the carrier scaffold can also be loaded with gelatin, collagen, fibroin, hydrogel or a combination thereof.

In another preferred embodiment, the chondrogenic culture is an in vitro culture using chondrogenic induction liquid.

In another preferred embodiment, the chondrogenic induction liquid comprises the following components: high glucose DMEM medium, 1% 1×ITS premix (ITS universal culture mixture containing insulin, transferrin, selenous acid, linoleic acid, bovine serum albumin, pyruvic acid, and ascorbic acid phosphate), 40 μg/ml proline, 10 ng/ml TGF-β1, 100 ng/ml IGF-1, 40 ng/ml dexamethasone and 50 μg/ml vitamin C.

In another preferred embodiment, the time for chondrogenic culture is 2-10 weeks, preferably 4-8 weeks, and most preferably 6 weeks.

In another preferred embodiment, the tissue engineering cartilage can be used for repairing joint defects and/or other types of hard tissue defects.

In the second aspect of the present invention, it provides a method for preparing the tissue engineering cartilage according to the first aspect of the present invention, comprising the steps of: inoculating a population of bone marrow mesenchymal stem cells into the carrier scaffold, and performing in vitro chondrogenic culture, thereby obtaining the tissue engineering cartilage.

In another preferred embodiment, the population of bone marrow mesenchymal stem cells is inoculated into the carrier scaffold by direct filling.

In another preferred embodiment, the population of bone marrow mesenchymal stem cells is inoculated into the carrier scaffold in the form of a suspension of bone marrow mesenchymal stem cells, with a concentration of 40-80×10⁶ cells/mL.

In another preferred embodiment, the method comprises the following specific steps:
(1) Extracting bone marrow fluid from a patient under sterile conditions, isolating, expanding and culturing bone marrow mesenchymal stem cells in vitro to P2-P5 passages, preferably P3 passage;
(2) Collecting and resuspending bone marrow mesenchymal stem cells, and inoculating them into a decalcified bone scaffold at a density of 40-80×10⁶ cells/cm³, followed by incubation for 2-4 hours;
(3) Adding chondrogenic induction liquid and performing in vitro chondrogenic culture for 2-10 weeks, preferably 4-8 weeks, and most preferably 6 weeks, thereby obtaining the tissue engineering cartilage.

In the third aspect of the present invention, it provides a use of the tissue engineering cartilage according to the first aspect of the present invention for the preparation of medical products for repairing joint defects or other types of hard tissue defects or deformities.

In another preferred embodiment, the joint defects comprise cartilage defects, hard bone defects, or combinations thereof.

In another preferred embodiment, the joint defect is a knee joint defect, an elbow joint defect, a hip joint defect, an ankle joint defect, a wrist joint defect, a mandibular joint defect, or a combination thereof.

In another preferred embodiment, the other types of hard tissue defects or deformities include, but are not limited to, tibial defects, femoral defects, humeral defects, mandibular deformities, and zygomatic deformities.

In the fourth aspect of the present invention, it provides a method for repairing joint defects or other types of hard tissue defects or deformities, comprising a step of using the tissue engineering cartilage according to the first aspect of the present invention and transplanting it into the tissue defect or deformity site to be repaired of a patient.

In another preferred embodiment, the joint defects comprise cartilage defects, hard bone defects, or combinations thereof.

In another preferred embodiment, the joint defect is a knee joint defect, an elbow joint defect, a hip joint defect, an ankle joint defect, a wrist joint defect, a mandibular joint defect, or a combination thereof.

In another preferred embodiment, the other types of hard tissue defects or deformities include, but are not limited to, tibial defects, femoral defects, humeral defects, mandibular deformities, and zygomatic deformities.

In another preferred embodiment, the transplantation is a minimally invasive transplantation, including arthroscopy or other minimally invasive implantation techniques.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the gross view and histological staining of the tissue engineering cartilage of the present invention; A1-A3: Gross view, the tissue engineering cartilage had an ivory white-like cartilage appearance; B1-B3: Hematoxylin-eosin (HE) staining, the staining results showed that the tissue engineering cartilage constructed in vitro had a typical cartilage cavity-like structure; C1-C3: Safranin O (SO) staining, the staining results showed that the tissue engineering cartilage constructed in vitro contained a large amount of cartilage specific extracellular matrix.

FIG. 2 shows the surgical procedures for the tissue engineering cartilage transplantation of the present invention; A: Selection of tissue engineering cartilage with appropriate size and shape; B: Observation of the damaged area of femoral cartilage under the microscope; C: After debridement of the damaged surface, placing the selected cartilage into the pre drilled hole; D: After repeated implantation at a certain density.

FIG. 3 shows the MRI examination results before and after the tissue engineering cartilage transplantation surgery; A (A1, A2) shows preoperative MRI findings, showing degenerative changes in the medial compartment of the joint, with obvious damage to the medial femoral cartilage and subchondral bone, accompanied by cystic degeneration; B (B1, B2) shows the MRI findings six months postoperatively, showing significant reduction in preoperative edema, good positioning of the graft, and continuous subchondral bone and cartilage signals, indicating good regeneration of the joint cartilage and bone.

### DETAILED DESCRIPTION

After extensive and in-depth research, the present inventor unexpectedly discovered and developed a tissue engineering cartilage based on a decalcified bone scaffold for the first time. In the present invention, decalcified bone matrix, a natural material with good mechanical strength, was used as a scaffold, and inoculated with autologous bone marrow mesenchymal stem cells from a patient to construct a tissue engineering cartilage in vitro. Then the tissue engineering cartilage was transplanted to the defect site by arthroscopic minimally invasive surgery, achieving effective repair and functional reconstruction of the patient's joint defect.

### Term

Unless otherwise defined, all technical and scientific terms used herein have the same meanings as those commonly understood by those skilled in the art to which the present invention belongs.

As used herein, the term “tissue-engineering cartilage” can be used interchangeably with “tissue engineering cartilage constructed based on a decalcified bone scaffold”, both referring to the bone marrow mesenchymal stem cell-decalcified bone scaffold composite as described herein, which may or may not have undergone in vitro chondrogenic culture.

As used herein, the term “inoculation” means the inoculation of bone marrow mesenchymal stem cells isolated from the patient's bone marrow fluid into a cell culture dish, and may also mean the inoculation of bone marrow mesenchymal stem cells that have been expanded and cultured in vitro to P4-P5 passages into a decalcified bone scaffold and evenly distributed therein. The meaning of “inoculation” as used can be understood by those skilled in the art from the context.

As used herein, when used in reference to specific enumerated values, the term “about” means that the value may vary by no more than 1% from the enumerated value. For example, as used herein, “about 100” includes all values between 99 and 101 (e.g., 99.1, 99.2, 99.3, 99.4, etc.).

As used herein, the terms “contain” or “include (or comprise)” can be open, semi-closed, or closed. In other words, the terms also include “substantially consisting of . . . ” or “consisting of . . . ”

### Bone Marrow Mesenchymal Stem Cells and Preparation Thereof

The bone marrow mesenchymal stem cells used in the present invention are isolated and expanded from the patient's autologous bone marrow fluid.

Specifically, 3-5 ml of bone marrow was aspirated from the patient's anterior superior iliac spine and placed on PercoII separation liquid (with a density of 1.073 g/L) for gradient density centrifugation. The ratio of bone marrow to separation liquid was 1:2. Centrifugation was performed at 2550 r/min for 30 minutes, then the fog-like cell layer in the middle was aspirated, and washed once with phosphate buffered saline (PBS). After centrifugation at 1550 r/min, the supernatant was discarded, then the nucleated cells were obtained and inoculated into the culture dish with a density of 2×10⁷ cells/cm² for in vitro cell expansion.

After 48 hours of primary cell inoculation, the medium was replaced. When the cells reached a fusion degree of 80% to 90%, they were digested with 0.25% trypsin, passaged at a density of 2×103 cells/cm², and cultured in an incubator at 37° C. with 5% CO₂ until P2-P5 passages. The cells were collected and counted to obtain a suspension of bone marrow mesenchymal stem cells suitable for inoculation.

### Decalcified Bone Scaffold

In a preferred embodiment of the present invention, the carrier scaffold is a decalcified bone scaffold (also known as a decalcified bone matrix) with a thickness of 0.3-0.8 cm, preferably 0.4-0.6 cm, and most preferably 0.5 cm. The decalcification amount of the decalcified bone matrix is 30% to 50%, with an appropriate degree of decalcification and good support effect, making it easy to be trimmed and shaped to a suitable shape and size. The pore diameter of the demineralized bone matrix is 200-400 μm, which is easy to be inoculated with bone marrow mesenchymal stem cells.

Decalcified bone matrix (DBM) is a tissue regeneration material with low immunogenicity that is obtained from allogeneic or xenogeneic bone by decalcification. It has good biological characteristics and biodegradability, promotes tissue regeneration, and can effectively repair various types of hard tissue injuries alone or in combination with autogenous bone, other biomaterials, and growth factors. It is an ideal tissue engineering scaffold material. However, the decalcified bone matrix itself lacks the ability for active tissue regeneration. When used alone, it will degrade and be absorbed, resulting in the inability to maintain the repair effect for a long time. The present invention uses bone marrow mesenchymal stem cells to be inoculated into decalcified bone scaffolds to construct tissue engineering cartilage, which can achieve stable tissue regeneration, defect repair, and functional reconstruction at joint defect sites.

### The Medium Used in the Present Invention

Bone marrow mesenchymal stem cell culture medium: The culture medium used for bone marrow mesenchymal stem cells contains 10 g of low glucose DMEM medium, 300 mg of L-glutamine, 50 mg of vitamin C, and 3.7g of sodium bicarbonate per liter of liquid. Preferably, 2-5 ng/ml of basic fibroblast growth factor (bFGF) is added.

Chondrogenic induction liquid: high glucose DMEM medium, 1% 1×ITS premix (ITS universal culture mixture containing insulin, transferrin, selenous acid, linoleic acid, bovine serum albumin, pyruvic acid, and ascorbic acid phosphate), 40 μg/ml proline, 10 ng/ml TGF-β1, 100 ng/ml IGF-1, 40 ng/ml dexamethasone, and 50 μg/ml vitamin C.

### HE Staining and Saf-O Staining

HE staining: hematoxylin-eosin staining, referred to as HE staining, is one of the commonly used staining methods in paraffin section technology. The hematoxylin dye solution is alkaline, which mainly makes the chromatin in the nucleus and the nucleic acid in the cytoplasm turn purple-blue; the eosin is an acid dye, which mainly makes the components in the cytoplasm and extracellular matrix turn red.

Saf-O staining: also known as safranine O staining, is a commonly used cartilage staining method. The principle of Saf-O staining is that the basophilic cartilage combines with the alkaline dye safranine O to present a red color. Safranine O is a cationic dye that combines with multiple anions. Its ability to stain cartilage tissue is based on the combination of cationic dyes with polysaccharide anionic groups (chondroitin sulfate or keratan sulfate).

### Tissue Engineering Cartilage of the Present Invention

In one aspect of the present invention, it provides a tissue engineering cartilage, which is a bone marrow mesenchymal stem cell-decalcified bone scaffold composite formed by inoculating bone marrow mesenchymal stem cells isolated and expanded in vitro into a carrier scaffold, and then subjecting them to in vitro chondrogenic culture. In a preferred embodiment of the present invention, the bone marrow mesenchymal stem cells are isolated from the patient's autologous bone marrow fluid and expanded and cultured in vitro to P2-P5 passages, and the carrier scaffold is a decalcified bone scaffold; The bone marrow mesenchymal stem cells are inoculated into the decalcified bone scaffold in the form of a cell suspension by direct filling, with a inoculation density of 20-100×10⁶ cells/cm³, preferably 40-80 x 106 cells/cm3; The decalcified bone scaffold loaded with bone marrow mesenchymal stem cells is cultured in chondrogenic induction liquid for in vitro chondrogenic culture, ultimately forming a tighter integrated structure of bone marrow mesenchymal stem cell-decalcified bone scaffold composite. The tissue engineering cartilage of the present invention has an ivory like cartilage appearance, and tissue staining shows that it not only has a typical cartilage cavity-like structure, but also contains a large amount of cartilage specific extracellular matrix. Due to the bidirectional differentiation potential of bone marrow mesenchymal stem cells into cartilage and bone, the tissue engineering cartilage of the present invention implanted into the defect site can be used to repair joint defects or other types of hard tissue defects or deformities, achieving integrated repair and functional reconstruction of cartilage-bone defects.

### The Main Advantages of the Present Invention

The present invention proposes a method for minimally invasive repair of joint defects using a tissue engineering cartilage constructed based on a decalcified bone scaffold, which has beneficial effects including:
(1) The mechanical strength of cartilage grafts constructed using existing tissue engineering techniques for repairing joint defects is extremely limited, failing to achieve immediate mechanical support at the defect site; However, the mechanical strength of the decalcified bone scaffold is excellent, and the tissue engineering cartilage constructed in vitro based on this type of scaffold has good mechanical properties, which can achieve immediate mechanical support at the defect site.
(2) Decalcified bone matrix, as a degradable natural material, can be degraded in vivo and has lower immunogenicity compared to artificially synthesized polymer scaffold materials.
(3) The existing methods of tissue engineering for repairing joint defects usually use chondrocytes to construct cartilage grafts for defect repair. This type of methods can only repair cartilage defects, but most joint defect patients are accompanied by varying degrees of subchondral bone damage, so the existing technology cannot achieve integrated joint cartilage-bone repair; The present invention uses autologous bone marrow mesenchymal stem cells from patients with bidirectional differentiation potential of cartilage and bone as seed cells to construct tissue engineering cartilage for defect repair, which can achieve integrated repair and functional reconstruction of joint cartilage-bone defects.
(4) Decalcified bone scaffolds have good malleability and scaffold materials can be customized according to the patient's defect area and shape to construct tissue engineering cartilage with a specific shape.
(5) The tissue engineering cartilage constructed using autologous bone marrow mesenchymal stem cells from patients in the present invention has better environmental tolerance compared to traditional stem cell-material composites, and is suitable for long-distance transportation. Therefore, it is conducive to the promotion and application of this technology.
(6) The seed cells used in the tissue engineering cartilage constructed by the present invention are autologous bone marrow mesenchymal stem cells from patients, which have ossification potential and thus can also be used for repairing other types of hard tissue defects. Hereinafter, the present invention will be further described with reference to specific examples. The present invention is further explained below in conjunction with specific example. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight. Unless otherwise stated, the materials and reagents used in the examples of the present invention are commercially available products.

### Example 1

### Preparation of the Tissue Engineering Cartilage Constructed Based on a Decalcified Bone Scaffold

(1) 3-5 ml of bone marrow was aspirated from the patient's anterior superior iliac spine and placed on PercoII separation liquid (with a density of 1.073 g/L) for gradient density centrifugation. The ratio of bone marrow to separation liquid was 1:2. Centrifugation was performed at 2550 r/min for 30 minutes, then the fog-like cell layer in the middle was aspirated, and washed once with phosphate buffered saline (PBS). After centrifugation at 1550 r/min, the supernatant was discarded, then the nucleated cells were obtained and inoculated into the culture dish with 2×10⁷ cells/cm² for in vitro cell expansion.
(2) After 48 hours of primary cell inoculation, the medium was replaced. When the cells reached a fusion degree of 80% to 90%, they were digested with 0.25% trypsin, passaged at a density of 2×10³ cells/cm², and cultured in an incubator at 37° C. with 5% CO₂ until P2-P5 passages. The cells were collected and counted.
(3) The bone marrow mesenchymal stem cells were collected and resuspended to prepare a cell suspension with a concentration of 40-80×10⁶ cells/mL, and inoculated into a cylindrical decalcified bone scaffold (diameter 4-8 mm, height 6-10 mm). After incubation for 2-4 hours, chondrogenic induction liquid was added to induce in vitro chondrogenesis for 4-8 weeks to construct tissue engineering cartilage.

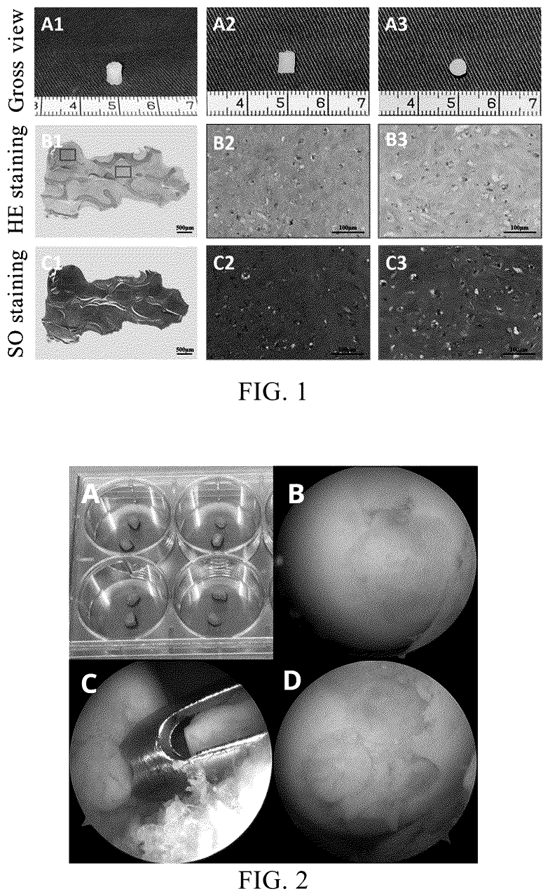

The tissue engineering cartilage prepared by the present invention had a typical ivory like appearance of cartilage tissue (FIG. 1, A1-A3), and histological examination showed that a large amount of cartilage specific extracellular matrix components was secreted, making it a typical cartilage tissue (FIG. 1, B1-B3, C1-C3).

### Example 2

### Minimally Invasive Repair of Joint Defects by Using Tissue Engineering Cartilage Constructed Based on a Decalcified Bone Scaffold

The patient underwent general anesthesia in a supine position. Using standard anterior medial and anterior lateral approaches to the knee joint, an arthroscopic minimally invasive cleaning was performed on the injured surface to the subchondral bone, and the tissue engineering cartilage prepared in Example 1 was transplanted to the defect site (FIG. 2).

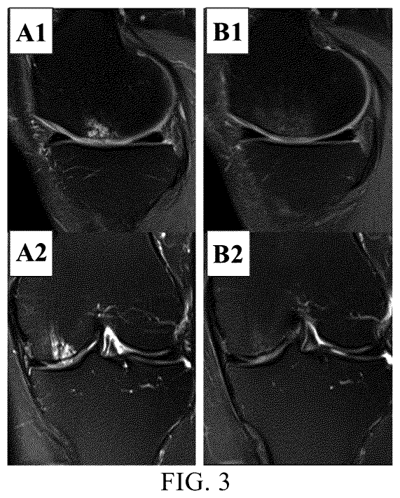

Preoperative MRI examination of the patient showed significant damage to cartilage and subchondral bone, accompanied by cystic degeneration and edema signals (FIG. 3, A). After transplantation, the graft was in good position, the edema area was significantly reduced, and continuous cartilage signals could be seen (FIG. 3, B).

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A tissue engineering cartilage, which comprises:
(a) a carrier scaffold comprising a decalcified bone scaffold; and
(b) bone marrow mesenchymal stem cells inoculated into or loaded onto the carrier.

2. The tissue engineering cartilage according to claim 1, wherein the tissue engineering cartilage comprises a complex formed by inoculating the bone marrow mesenchymal stem cells into the carrier and subjecting them to chondrogenic culture; in the complex, the bone marrow mesenchymal stem cells are loaded onto the carrier and form a tighter integrated structure with the carrier.

3. The tissue engineering cartilage according to claim 1, wherein the bone marrow mesenchymal stem cells are bone marrow mesenchymal stem cells cultured in vitro to P2 to P5 passages, preferably P3 passage bone marrow mesenchymal stem cells.

4. The tissue engineering cartilage according to claim 2, wherein the inoculation density of the bone marrow mesenchymal stem cells into the carrier is 20-100×10⁶ cells/cm³, preferably 40-80×10⁶ cells/cm³.

5. The tissue engineering cartilage according to claim 2, wherein the chondrogenic culture is an in vitro culture using chondrogenic induction liquid.

6. The tissue engineering cartilage according to claim 6, wherein the chondrogenic induction liquid comprises the following components: high glucose DMEM medium, 1% 1×ITS premix (ITS universal culture mixture containing insulin, transferrin, selenous acid, linoleic acid, bovine serum albumin, pyruvic acid, and ascorbic acid phosphate), 40 μg/ml proline, 10 ng/ml TGF-β1, 100 ng/ml IGF-1, 40 ng/ml dexamethasone and 50 μg/ml vitamin C.

7. The tissue engineering cartilage according to claim 2, wherein the time for chondrogenic culture is 2-10 weeks, preferably 4-8 weeks, and most preferably 6 weeks.

8. A method for preparing the tissue engineering cartilage according to claim 1, comprising the steps of: inoculating a population of bone marrow mesenchymal stem cells into the carrier scaffold, and performing in vitro chondrogenic culture, thereby obtaining the tissue engineering cartilage.

9. The method according to claim 8, wherein the population of bone marrow mesenchymal stem cells is inoculated into the carrier scaffold in the form of a suspension of bone marrow mesenchymal stem cells, with a concentration of 40-80×10⁶ cells/mL.

10. A medical product comprising the tissue engineering cartilage according to claim 1.
